(19) United States
(12) Patent Application Publication
(10) Pub. No.: US 2007/0233208 A1
(43) Pub. Date: Oct. 4, 2007

(54) Light therapy bandage with imbedded emitters

(75) Inventors: Andrew F. Kurtz, Macedon, NY (US); James E. Roddy, Rochester, NY (US); Mark E. Bridges, Spencerport, NY (US); Paul R. Switzer, Batavia, NY (US); Roger H. Connelly, Hilton, NY (US)

Correspondence Address: PATENT LEGAL STAFF;EASTMAN KODAK COMPANY, 343 STATE STREET, ROCHESTER, NY, 14650-2201, US

(73) Assignee: Eastman Kodak Company

(21) Appl. No.: 11/390,862

(22) Filed: Mar. 28, 2006

(51) Int. Cl. A61N 5/06

(52) U.S. Cl. 607/88

(57) ABSTRACT

A light therapy bandage (300) for treating medical conditions comprises a plurality of flexible sheet circuitry (350), each of which is fabricated with a serpentine pattern provided with one or more surface mounted light emitting devices (372). A flexible transparent material (470) included within the substrate (410) and the surface mounted light emitting devices are imbedded in the flexible transparent material. A semi-permeable transparent membrane (450) controls the flow of moisture and moisture vapor to and from the tissues (200). A plurality of vapor channels (460) extend from the semi-permeable transparent membrane and through the substrate.

### CROSS REFERENCE TO RELATED APPLICATIONS

Reference is made to commonly-assigned copending U.S. patent application Ser. No. 11/087,300 filed Mar. 23, 2005, entitled LIGHT GUIDE BANDAGE, by Olson et al., the disclosure of which is incorporated herein.

### FIELD OF THE INVENTION

The invention relates generally to a light therapy device and in particular, to a light therapy device for use in close proximity, or in contact with, the skin or a patient.

### BACKGROUND OF THE INVENTION

The term “phototherapy” relates to the therapeutic use of light, and the term “illuminator” or “light therapy device” or “phototherapy device” refers to a device that is generally intended to be used externally to administer light to the skin of a patient for therapeutic purposes.

External light therapy has been shown to be effective in treating various medical conditions, for example, seasonal affective disorder, psoriasis, acne, and hyperbilirubinemia common in newborn infants. Light therapy has also been employed for the treatment of wounds, burns, and other skin surface (or near skin surface) ailments. As one well-known example, light therapy can be used to modify biological rhythms in humans, such as circadian (daily) cycles that affect a variety of physiologic, cognitive, and behavioral functions. Light therapy has also been used for other biological treatments that are less recognized. For example, in the late 1800's, Dr. Niels Finsen found that exposure to ultraviolet radiation aggravated smallpox lesions. Thus, he illuminated his patients with light with the UV filtered out. Dr. Finsen further discovered that exposure with the residual red light sped healing in recovering smallpox victims. Finsen also determined that ultraviolet radiation could be used to heal tuberculosis lesions. As a result, in 1903, Dr. Finsen was awarded a Nobel Prize for his use of red light therapy to successfully treat smallpox and tuberculosis.

In the 1960's and 1970's researchers in Eastern Europe undertook the initial studies that launched modern light therapy. One such pioneer was Endre Mester (Semmelweiss Hospital, Budapest, Hungary), who in 1966, published the first scientific report on the stimulatory effects of non-thermal ruby laser light (694 nm) exposure on the skin of rats. Professor Mester found that a specific range of exposure conditions stimulated cell growth and wound healing, while lesser doses were ineffective and larger doses were inhibitory. In the late 1960's, Professor Mester reported the use of laser light to treat non-healing wounds and ulcers in diabetic patients. Mester's 70% success rate in treating these wounds lead to the development of the science of what he called “laser biostimulation.”

Photodynamic therapy (PDT) is one specific well-known example of light therapy, in which cancerous conditions are treated by a combination of a chemical photo-sensitizer and light. Typically in this instance, several days before the light treatment, a patient is given the chemical sensitizer, which generally accumulates in the cancerous cells. Once the sensitizer concentrations in the adjacent non-cancerous cells falls below certain threshold levels, the tumor can be treated by light exposure to destroy the cancer while leaving the non-cancerous cells intact.

As compared to PDT, light therapy, as exemplified by Professor Mester's pioneering work, involves a therapeutic light treatment that provides a direct benefit without the use of enabling external photo-chemicals. Presently, there are over 30 companies world wide that are offering light therapy devices for a variety of treatment applications. These devices vary considerably, with a range of wavelengths, power levels, modulation frequencies, and design features being available. In many instances, the exposure device is a handheld probe, comprising multitude light emitters; that can be directed at the patient during treatment. The light emitters, which typically are laser diodes, light emitting diodes (LEDs), or combinations thereof, usually provide light in the red-IR (˜600-1200 nm) spectrum, because the tissue penetration is best at those wavelengths. In general, both laser light and incoherent (LED) light seem to provide therapeutic benefit, although some have suggested that lasers may be more efficacious. Light therapy is recognized by a variety of terms, including low-level-laser therapy (LLLT), low-energy-photon therapy (LEPT), and low-intensity-light therapy (LILT). Despite the emphasis on “low” in the naming, in actuality, many of the products marketed today output relatively high power levels, of up to 1-2 optical watts. Companies that presently offer light therapy devices include Thor Laser (United Kingdom), Omega Laser Systems ((United Kingdom), MedX Health (Canada), Quantum Devices (United States), and Lumen Photon Therapy (United States).

Many different examples of light therapy and PDT devices are known in the patent art. Early examples include U.S. Pat. No. 4,316,467 (Muckerheide) and U.S. Pat. No. 4,672,969 (Dew). The most common device design, which comprises a hand held probe, comprising at least one light emitter, but typically dozens (or even 100) emitters, that is attached to a separate drive controller, is described in numerous patents, including U.S. Pat. Nos. 4,930,504 (Diamantapolous et al.); U.S. Pat. No. 5,259,380 (Mendes et al.); U.S. Pat. No. 5,464,436 (Smith); U.S. Pat. No. 5,634,711 (Kennedy et al.); U.S. Pat. No. 5,660,461 (Ignatius et al.); U.S. Pat. No. 5,766,233 (Thiberg); and U.S. Pat. No. 6,238,424 (Thiberg).

One shortcoming of the probe type laser therapy device is that it requires the clinician, or perhaps the patient, to actively apply the laser light to the tissue. Typically, the clinician holds the light therapy probe, aims the light at the tissue, and operates the device according to a treatment protocol. As a result, the laser therapy devices are often designed to emit high light levels, in order to reduce the time a clinician spends treating an individual patient to a few minutes or less, whether the application conditions are optimal or not. Additionally, in many such cases, the patient is required to travel to the clinician's facility to receive the treatment. Because of this inconvenience, patients are typically treated only 1-3 times per week, even if more frequent treatments would be more efficacious.

Certainly, these shortcomings with the handheld probes have been previously identified. For example, Laser Force Therapy (Elizabeth, Colo.) offers a disk-shaped probe (the “Super Nova”) that can be strapped onto the patient. While this is a potential improvement, the device does not conform to the shape of the tissue being treated. As an alternate approach, a variety of self-emissive light bandages have been suggested, in which a conformal pad having a light emitting inner surface is strapped directly on the patient. Since the patient can wear the device, perhaps under their clothes for a prolonged period of time, the convenience limitations of the handheld probe may be overcome.

Therapeutic light pads have been developed using woven bundles of optical fibers. Such devices are typically marketed for use in treating jaundice in infants. One example is the Biliblanket Plus, offered by Ohmeda Medical (Baltimore, Md.), which uses a high intensity halogen lamp, mounted in a controller and light coupled into a fiber bundle. The fiber bundle, nominally comprising 2400 individual optical fibers, is configured into a woven pad, in which the bends in the optical fibers cause local breakdown in total internal reflection, so that light is coupled out of the fiber over the full surface area of the pad. The general concept is shown in FIG. 1, wherein a light therapy device 50 comprises a woven fiber-optic pad 10 connected by a fiber-optic cable 12 to a controller 20 with an enclosure 14 for a source of light. The fiber-optic cable 12 has a protective coating of a plastic material such as vinyl and contains a plurality of individual optical fibers, not shown in FIG. 1, which transmit the light from the enclosure 14 to the woven fiber-optic pad 10 for emission toward the infant. A connector 16, affixed to an end of fiber-optic cable 12, positions the cable to receive light energy from a light source (internal to enclosure 14 and not shown). Another company, Respironics (Murrysville, Pa.), offers a similar system, the Wallaby Phototherapy System, for neonatal care of jaundice. The basic concept for a woven fiber-optic illuminator is described in U.S. Pat. No. 4,234,907 (Daniel). This type of medical light therapy pad is also described in prior art patents U.S. Pat. No. 5,339,223 (Kremenchugsky et al.) and U.S. Pat. No. 5,400,425 (Nicholas et al.), both assigned to Ohmeda Inc. While these devices are useful, they have limited utility and again are not optimized for wound care.

Alternately, light therapy devices have been described that use discrete light emitters fabricated into a dressing or bandage. As a first example, U.S. Pat. No. 6,569,189 (Augustine et al.) provides a heat therapy bandage that uses IR blackbody radiation generated from electrical resistance in circuit trace within the bandage. In this case, since the emitted light is broadband IR (nominally 3-30 microns), this bandage does not enable the use of specific illumination optical wavelengths that have been suggested to be optimal for treating various conditions. In particular, the wavelengths provided by this device may not advantageously activate the known photo-acceptor molecules in cells. Moreover, this device does not offer a means to vary the light spectrum in any useful way, nor is it optimized for wound treatment.

As a second example, Omnilight (Albuquerque, N. Mex.) offers the Versalight pads, which combine a controller (such as the VL3000) with a pad, wherein the pads comprise a multitude of discrete LEDs imbedded in a neoprene-covered foam. Bioscan Inc. (Albuquerque, N. Mex.) offers a similar suite of products for veterinary applications. In both cases, the products typically comprise a mix of IR and red LED emitters, arranged in a pattern across the pad. These devices are described in U.S. Pat. No. 4,646,743 (Parris), which teaches conformal pad light therapy devices in which an array of diodes is imbedded in pliable foam. These devices have greater flexibility than the prior one, but are again not optimized for wound treatment.

As an alternate approach, there are a variety of technologies being developed that involve self-emissive devices, rather than employing discrete emitters imbedded in a substrate. For example, devices have been described that use organic light emitting diodes (OLEDs), polymer light emitting diodes (P-LEDs), and thin film flexible electroluminescent sources (TFELs). As an example, U.S. Pat. No. 6,096,066 (Chen et al.) teaches a flexible LED array on a thin polymer substrate, with addressable control circuitry, slits for perspiration, and the use of LEDs, which could be replaced with OLEDs. Similarly, U.S. Pat. No. 6,866,678 (Shenderova) discloses a thin film electroluminescent (TFEL) phototherapy device based on high field electroluminescence (HFEL) or OLED technologies. Certainly, light therapy bandages based on these technologies have several potential advantages, including volume production, readily customizable temporal and spatial control from the addressing circuitry, and a very thin from factor, which could help conformability. However, even in the display markets (laptop computers, television, etc.), which is the primary target market, OLED technologies are not yet sufficiently mature to support volume production. Also, while self emissive light bandages will not be encumbered by lifetime issues and the resolution requirements imposed on the display market, such bandage type devices will have their own issues (minimizing toxicity, handling moisture, and providing sufficient output power or IR output light) that will likely affect the appearance of such devices in health markets.

Thus, a design approach based on the use of discrete emitters, and generally similar to that described in U.S. Pat. No. 4,646,743, may be a best approach for achieving a light therapy bandage. Several other device designs beyond that of U.S. Pat. No. 4,646,743 are known in the prior art, including:
U.S. Pat. No. 5,358,503 (Bertwell et al.), which provides a conformal pad utilizing tightly packed LEDs and adjacent resistors, which is placed in contact with the tissue, so as to provide both light and thermal treatments.
U.S. Pat. No. 5,913,883 (Alexander et al.), which provides a conformal therapeutic facial mask comprising a plurality of LEDs held off of the tissue by spacer pads.
U.S. Pat. No. 6,096,066 (Chen) provides a conformal light therapy patch having addressable LEDs interconnected by control circuitry and having perspiration slits.
U.S. Pat. No. 6,443,978 (Zharov) describes a conformal light source array device that has spacer layers to hold the emitters off the tissue, bio-sensors, arid magnetic stimulators.
Other prior art references that provide for conformal light therapy devices with discrete light emitters mounted to a substrate include U.S. Pat. No. 6,187,029 (Shapiro et al.), U.S. Patent Application Publication No. 2005/0187597 (Vanderschuit), and U.S. Patent Application Publication No. 2005/0177093 (Barry et al.).

However, none of the above prior art references discuss light therapy devices that were designed with a real potential to function as a light therapy bandage or dressing, with potential applicability to wound care. By comparison, a prior design described in U.S. Pat. No. 5,616,140 (Prescott) provides a conformal light therapy device 50 comprising light emitters 75 and flexible drive circuitry 85 fabricated within a multi-layer pad or bandage 55. An illustration of the device 50 of U.S. Pat. No. 5,616,140 is shown in FIG. 2a. This device has several useful features, including an onboard battery 90, a molded silicone housing 65 with clear windows 80, heat sinks 95, and attachment straps 60, but the design is not optimized for large area conformability, operational temperature, or for wound care.

As another example, U.S. Pat. No. 6,743,249 (Alden), as shown in FIG. 2b describes a light therapy treatment device 50 with a controller (not shown) having a multitude of interconnected light emitters 75 mounted in a shell 105, with a surrounding liner 110 and a heat dissipating layer 100. The shell 105 is described as comprising a molded and cured liquid silicone rubber material, which is generally flexible, while the liner 110 nominally comprises a transparent tacky silicone gel material, which provides a tacky surface 120 that is placed in contact with the skin. Liner 100 can also contain an optical diffuser 115. FIG. 2c shows an alternate light therapy device 50, described in U.S. Pat. No. 6,290,713 (Russell), in which a light therapy device 50 (controller not shown) comprises a pad with a series of light emitters 75 imbedded in a structure between front cover 145 and back cover 147. Substrate 130 can include an internal reflector 135 and flexible circuitry, while front cover 145 can be fabricated with imbedded bubbles or beads 140 (for light diffusion). The pad or bandage 55 is also equipped with cooling channels 155 and secondary cooling channels 157 to help dissipate the heat generated by light emitters 75. The light emitters 75 can be surface mount devices.

Although these various patents include many interesting elements, none of them have really presented a design for a light therapy bandage or dressing that is sufficiently conformal to be applied in close contact to the complex three-dimensional shapes present on the human body, such as the sole of the foot, or the lower back. Additionally, there are opportunities to improve the heat management of this type of device. Finally, there are opportunities to improve the design of this type of device relative to the potential use as a primary or secondary wound care dressing.

### SUMMARY OF THE INVENTION

Briefly, according to one aspect of the present invention a light therapy bandage for delivering light energy to treat medical conditions in tissues includes a plurality of flexible sheet circuitry, each of which is fabricated with a serpentine pattern and each of which is provided with one or more surface mounted light emitting devices that emit the light energy. The flexible sheet circuitry is assembled into a substrate. A flexible transparent material included within the substrate is applied in such a way that the surface mounted light emitting devices are imbedded in the flexible transparent material. A semi-permeable transparent membrane is attached to the flexible transparent material, which controls the flow of moisture and moisture vapor to and from the tissues. A plurality of vapor channels extend from the semi-permeable transparent membrane and through the substrate. The light energy passes through the substrate and the semi-permeable membrane to be incident to the tissues, and the moisture vapor passes through the semi-permeable membrane and the vapor channels and into the surrounding environment.

### BRIEF DESCRIPTION OF THE DRAWINGS

The foregoing and other objects, features, and advantages of the invention will be apparent from the following more particular description of the embodiments of the invention, as illustrated in the accompanying drawings. The elements of the drawings are not necessarily to scale relative to each other.

FIG. 1 shows a perspective view of a prior art light therapy device comprising a fiber-optic mat type illuminator and a drive unit.

FIG. 2a, 2b, and 2c shows cross sectional side views of prior art diode-based light therapy bandages.

FIG. 3 shows a picture of human tissue having a chronic wound.

FIGS. 4a, 4b, and 4c show top views of the light therapy device of the present invention, with different configurations of light application.

FIGS. 5a, 5b, and 5c show cross sectional representative side views of wounds in combination with a light therapy wound dressing device of the present invention.

FIG. 6 shows a schematic view of a light therapy bandage of the present invention, showing an overall system configuration.

FIG. 7a shows schematic top and side views of a light therapy bandage of the present invention.

FIG. 7b shows a schematic top view of an alternate top view bandage of the present invention.

FIG. 7c shows two different schematic side views of a light therapy bandage of the present invention.

FIG. 7d shows a schematic top view of an alternate top view bandage of the present invention.

FIG. 7e shows a schematic top view of an alternate top view bandage of the present invention.

FIG. 8a shows a schematic side view of a light therapy bandage of the present invention.

FIG. 8b shows schematic top view of a light therapy bandage of the present invention

FIG. 9 shows a diagrammatic view of a circuitry design of the present invention.

FIGS. 10a-10c show several possible drive waveforms for operating the light therapy device of the present invention.

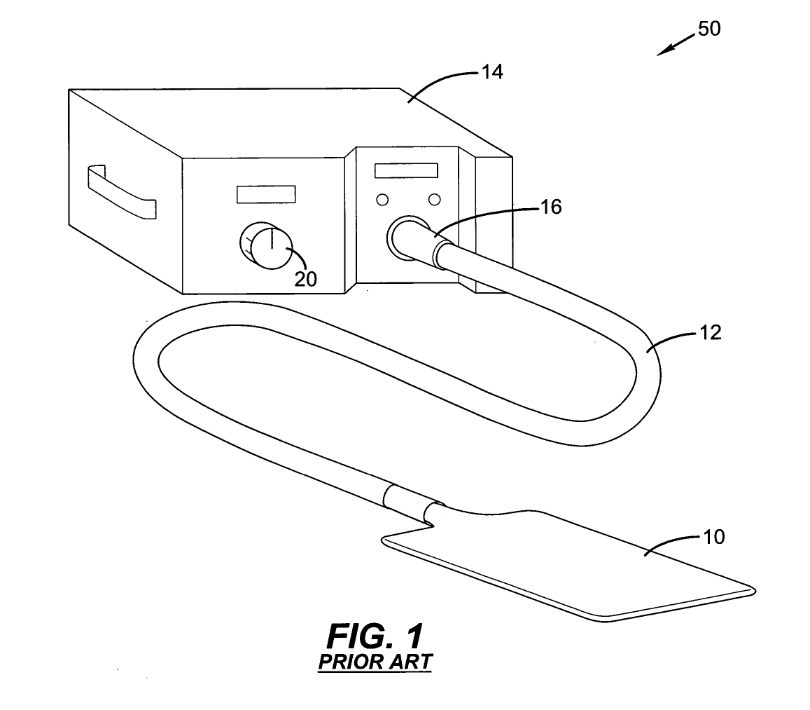

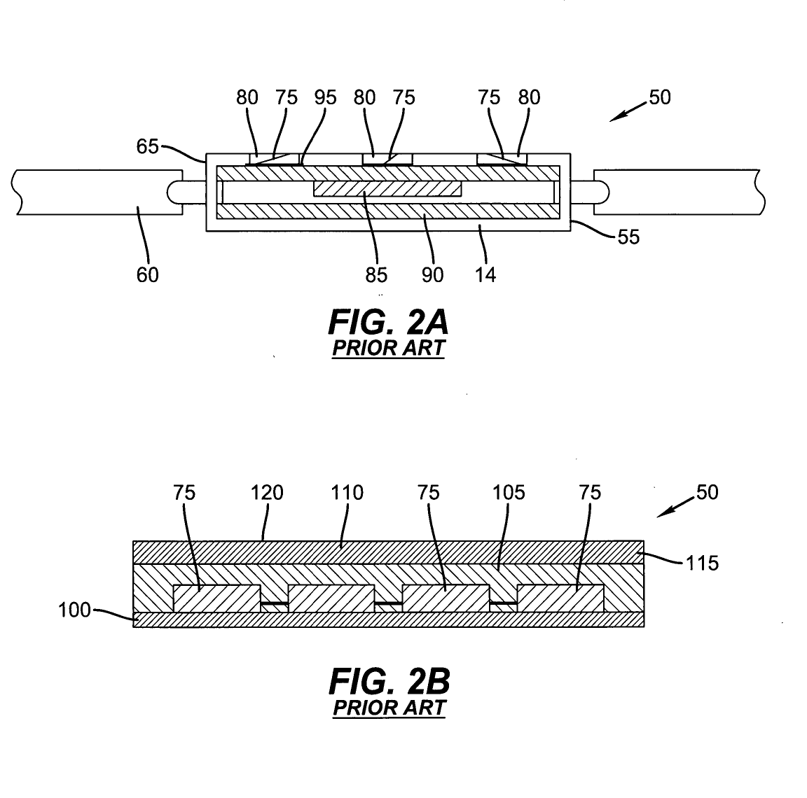

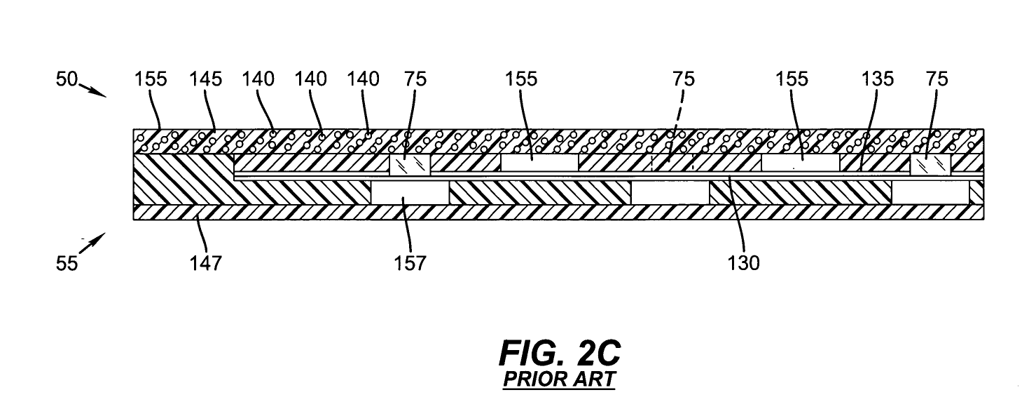

### DETAILED DESCRIPTION OF THE INVENTION

The following is a detailed description of the preferred embodiments of the invention, reference being made to the drawings in which the same reference numerals identify the same elements of structure in each of the several figures.

The present invention provides a flexible light therapy device having a plurality of applications, including but not limited to, the treatment of seasonal affective disorder, psoriasis, acne, diabetic skin ulcers, pressure ulcers, PDT, and hyperbilirubinemia common in newborn infants. The present invention delivers light energy by means of a flexible member that can be placed in contact with the skin of a patient. The present invention comprises a light therapy bandage or dressing, comprising a multitude if light emitters assembled within the bandage, such that the light is then incident onto the tissue. The device is nominally designed to be readily worn by the patient for a prolonged time period, and is potentially disposable thereafter.

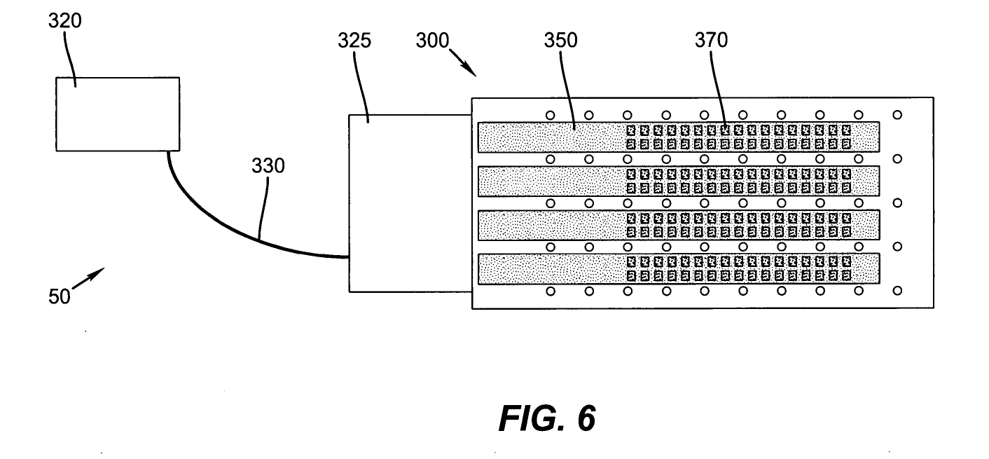

The basic device is shown in FIG. 6. Light therapy device 50 comprises a bandage 300 driven by a controller 320 that interacts the bandage via connective circuitry (or a wireless link) 330. Controller 320 facilitates the setting of treatment parameters such as light intensity, frequency, wavelength, modulation, and repeat treatment timing. Electrical power to drive the light emitting diodes 370 can also be supplied through controller 320, via connective circuitry 330. Controller 320 may also be incorporated directly within intermediate 325 or bandage 300, if it can be sufficiently simplified.

The light therapy bandage 300 is generally intended to have a modular design that would enable flexible patterns of use. For example, it may desirable for the light therapy bandage to be left in place on the patient between treatments. In that instance, the bandage may have an intermediate portion 325, which provides the immediate electrical connection to the bandage 300. The intermediate 325 could have a robust, low profile coupling means, so that the intermediate portion 325 and bandage 300 can be comfortably worn, potentially with pressure applied, during a prolonged (30 minutes, for example) treatment period. Alternately, the entire bandage 300 could be detached from the patient between treatments. For example, bandage 300 could have an attachment means, such as Velcro straps (not shown), to hold it in place around a limb.

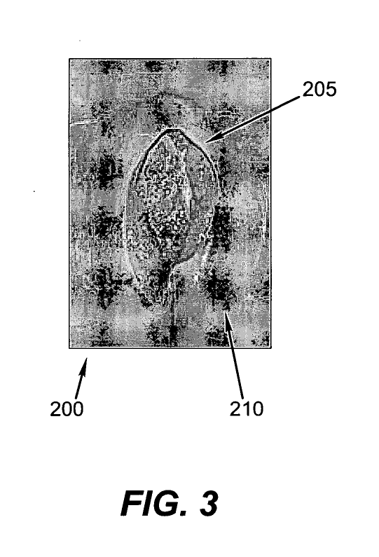

As an intermediate, bandage 300 could have a portion, including attachment points, that stays on the patient for an extended time (such as days), while another portion bearing the light emitting diodes 370 is removed between treatments. For example, a patient could receive periodic light therapy treatments for muscle pain and have the entire device removed between treatments. On the other hand, it is good practice relative to the treatment of wounds (see FIG. 3) to minimize disturbances to the healing wound site. In such cases it may be desirable for bandage 300 to be as conformal and comfortable as possible, that it can be left in-situ indefinitely (or at least for several days). The aforementioned design approach emphasizing modularity could also work. The use of a detachable intermediate connector 325 is an example of this approach, which has the added advantage that bandage 300 and intermediate 325 could both be contaminated (for example by wound exudates) without controller 320 being impacted.

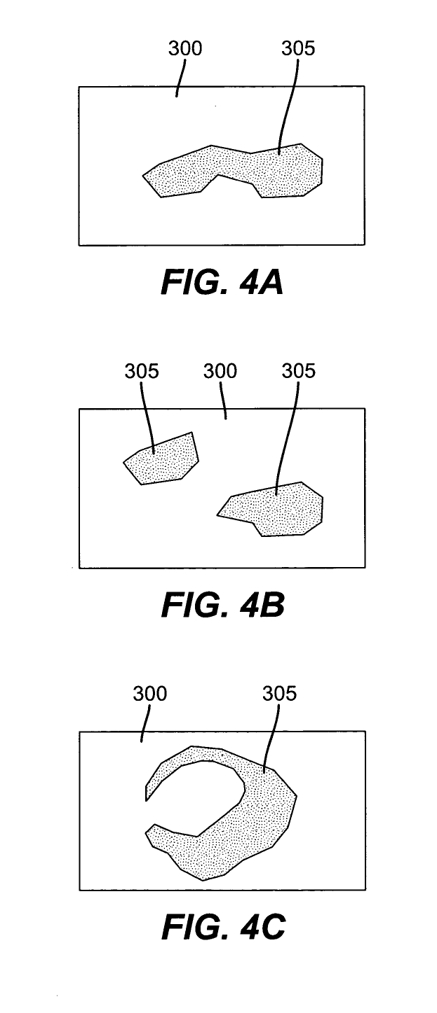

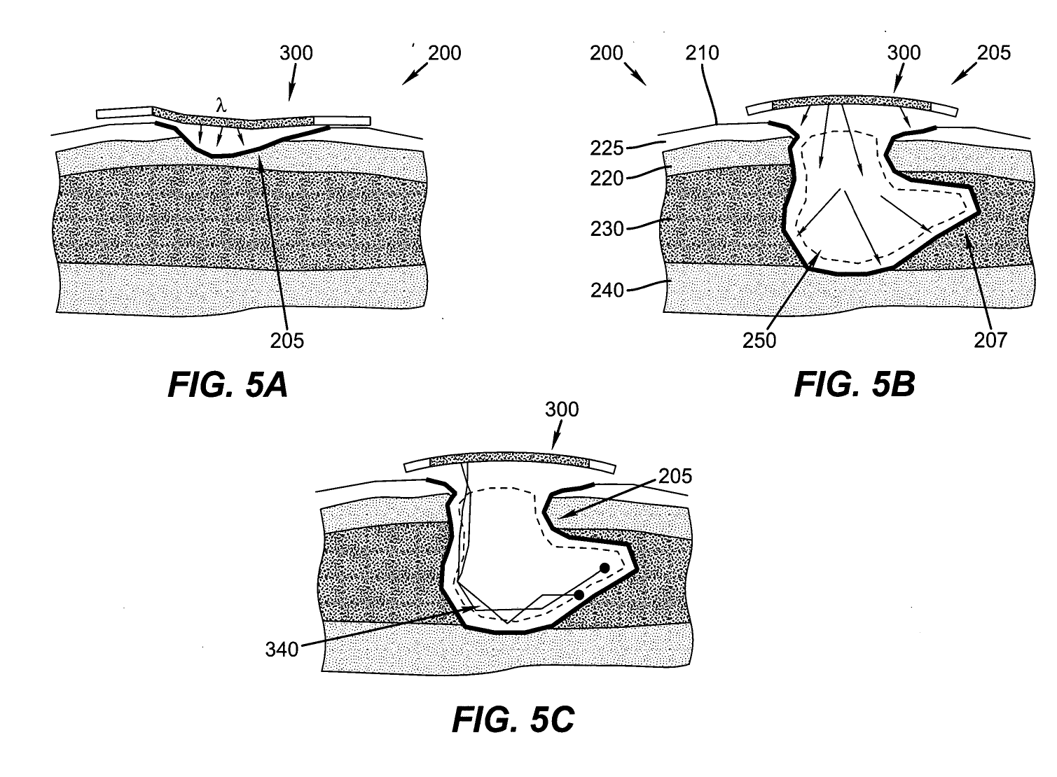

The general concept of the use of the present invention is depicted in FIG. 5a. A light therapy bandage 300 is used to apply light (λ) to a wound 205 in a tissue 200. The therapeutic light can be of one or more wavelengths in the ultraviolet, visible, or near infrared spectra, but is preferably red or near infrared light (600-1300 nm). As an area of tissue may have one or more adjacent wounds of different configurations, then one or more treatment areas 305, as generally depicted in FIGS. 4a-4c, may receive treatment from light therapy device 300.

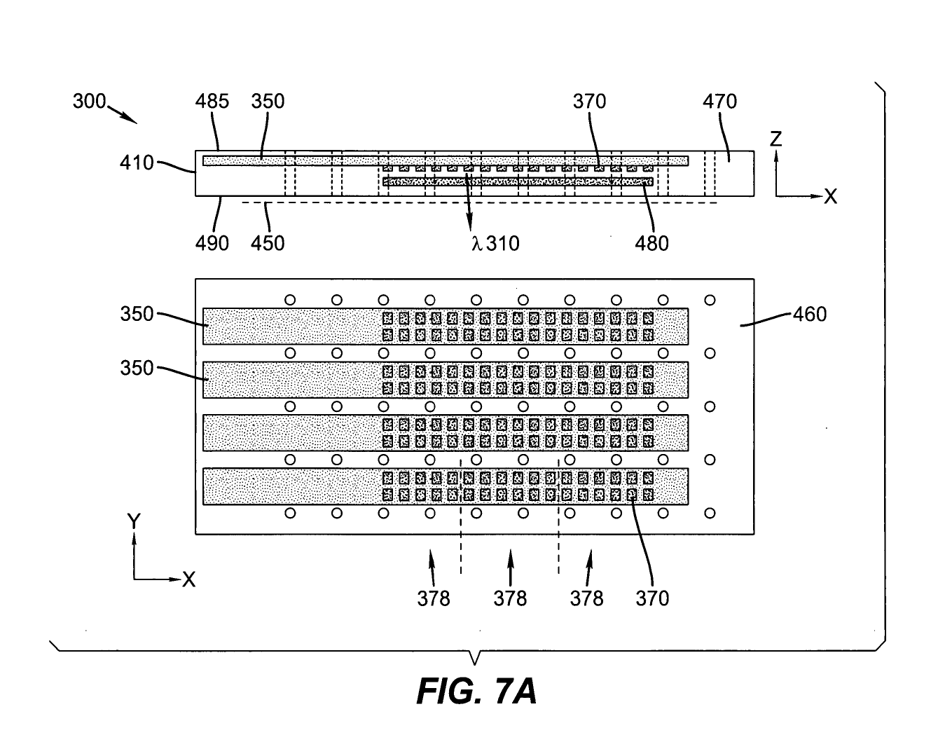

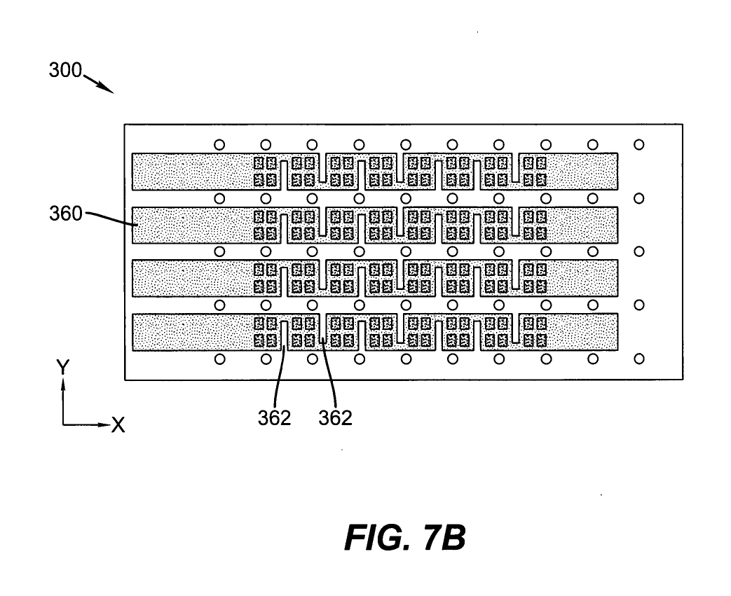

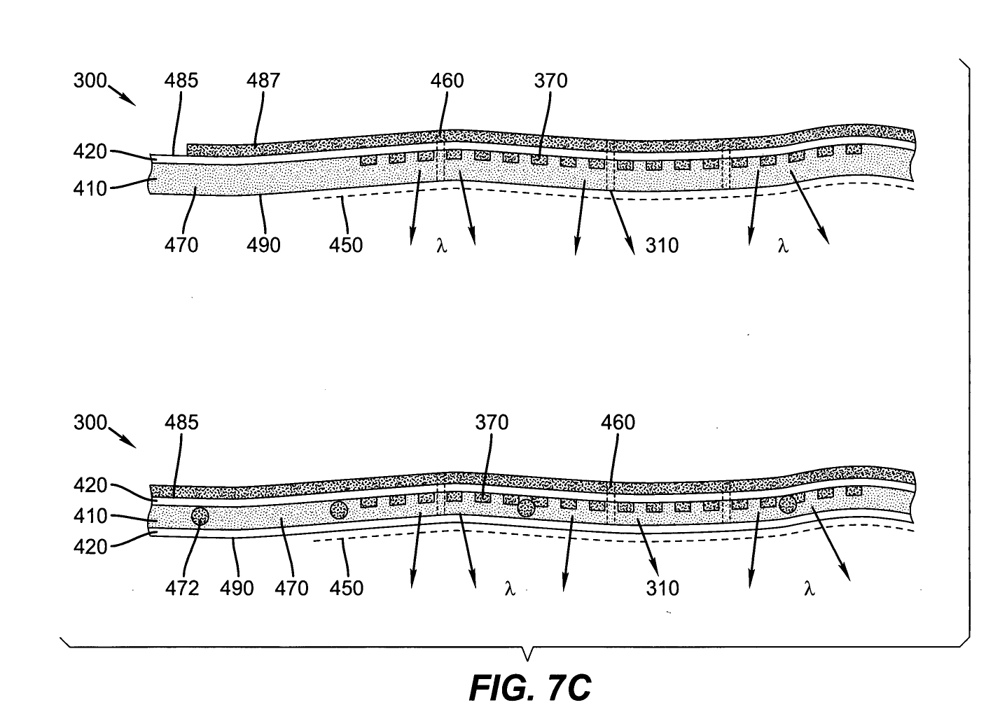

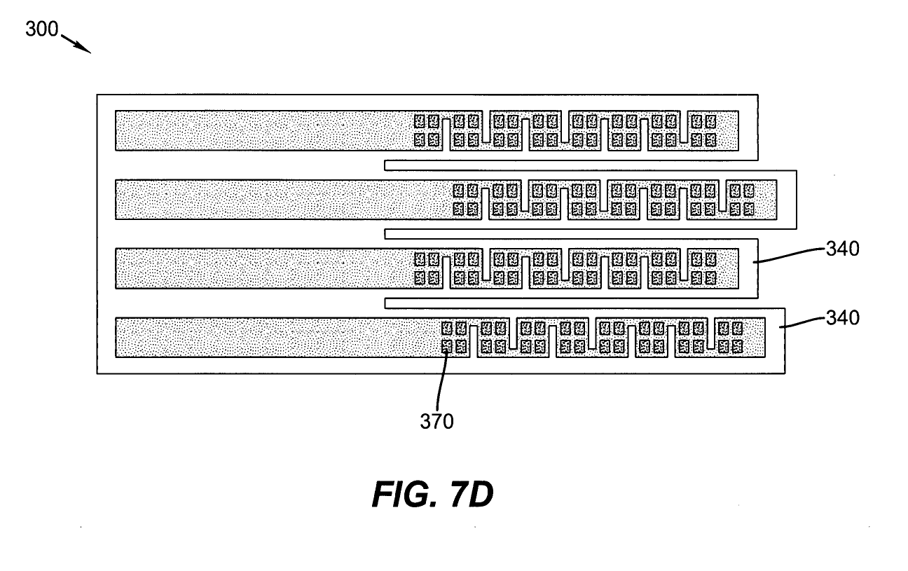

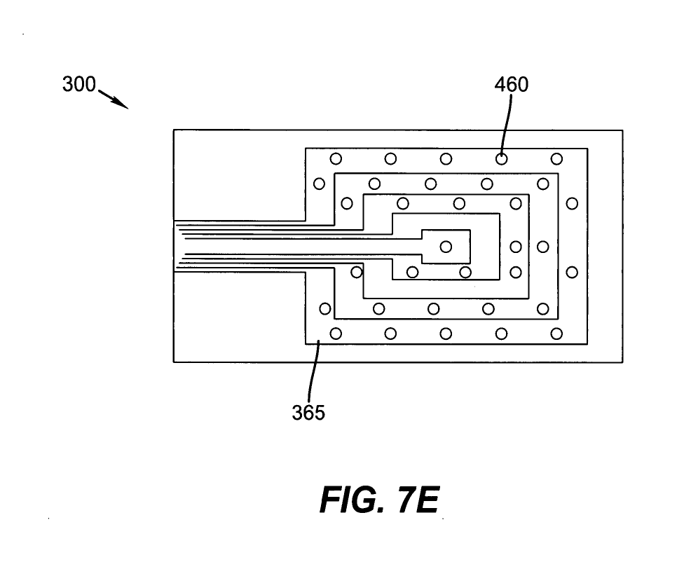

A more detailed view of light therapy bandage 300 is shown in FIG. 7a. The device nominally comprises a substrate 410 that has flex circuitry 350, bearing light emitting diodes 370, imbedded within it. The diodes 370 emit therapeutic light 310 that can be directed onto the tissue being treated (not shown). The substrate 410 includes a transparent material 470 between the light emitting diodes 370 and the exit surface 490. This material could be sheet polymer material, such as a polyurethane, or alternately a gel or foam material. An optical diffuser 480 may also be provided within the substrate 410. Light therapy bandage 300 also nominally includes a barrier membrane 450, which is attached to substrate 410, and vapor channels 460 which can be provided transversely through substrate 410.

It should be understood that the cross-sectional and top views of FIG. 7a are meant to be illustrative of the general concepts, and do not represent the actual relative physical size of the various constituent layers and components. Other figures are intended to be similarly illustrative.

Although the device could be used to treat multiple conditions, the concept is principally linked to the treatment of wounds. Wounds are characterized in several ways; acute wounds are those that heal normally within a few weeks, while chronic wounds are those that linger for months or even years. Wounds that heal by primary union (or primary intention) are wounds that involve a clean incision with no loss of substance. The line of closure fills with clotted blood, and the wound heals within a few weeks. Wounds that heal by secondary union (or secondary intention) involve large tissue defects, with more inflammation and granulation. Granulation tissue is needed to close the defect, and is gradually transformed into stable scar tissue. Such wounds are large open wounds as can occur from trauma, burns, and pressure ulcers. While surgical wounds are typically stitched or stapled shut, which reduces the burden on the wound dressing, either a subsequent infection or wound geometry can shift the burden. While such a wound may require a prolonged healing time, it is not necessarily chronic.

A chronic wound is a wound in which normal healing is not occurring, with progress stalled in one or more of the phases of healing. A variety of factors, including age, poor health and nutrition, diabetes, incontinence, immune deficiency problems, poor circulation, and infection can all cause a wound to become chronic. Typical chronic wounds include pressure ulcers, friction ulcers, and venous stasis ulcers. Stage 3 and Stage 4 pressure ulcers (see FIG. 3) are open wounds 205 that can occur whenever prolonged pressure is applied to skin 210 and tissues 200 covering bony outcrops of the body. Chronic wounds are also categorized, according to the National Pressure Ulcer Advisory Panel (NPUAP) relative to the extent of the damage:
Stage 1—has observable alteration of intact skin with changes in one or more of skin temperature, tissue consistency, or sensation (pain, itching). Pro-active treatment of Stage 1 and Pre-Stage 1 (also known as Stage 0) wounds could be beneficial.
Stage 2—involves partial thickness skin loss involving epidermis, dermis, or both. The ulcer is superficial and appears as an abrasion, blister, or shallow crater, much as depicted in FIG. 5a, where wound 205 penetrates the skin surface 210 and stratum corneum 225 and the epidermis 220.
Stage 3—Full thickness skin loss with damage or necrosis of subcutaneous tissue. FIG. 5b is generally illustrative of this type of wound, with wound 205 penetrating the epidermis 220 and the dermis 230, as well as a portion of the subcutaneous tissue 240.
Stage 4—Full thickness skin loss with extensive destruction, tissue necrosis, and damage to muscle, bone, or supporting structures (tendon, joint, capsule, etc.). Successful healing of Stage 4 wounds still involve loss of function (muscles and tendons are not restored).
Stage 5—Surgical removal of necrotic tissue usually required, and sometimes amputation. Death usually occurs from sepsis.

Wound healing also progresses through a series of overlapping phases, starting with coagulation (haemostasis), inflammation, proliferation (which includes collagen synthesis, angiogenesis, epithelialization, granulation, and contraction), and remodeling. Haemostasis, or coagulation, is the process by which blood flow is stopped after the initial wounding, and results in a clot, comprising fibrin, fibronectin, and other components, which then act as a provisional matrix for the cellular migration involved in the later healing phases. Many of the processes of proliferation, such as epithelialization and angiogenesis (creation of new blood vessels) require the presence of the extracellular matrix (ECM) in order to be successful. Fibroblasts appear in the wound during that late inflammatory phase (˜3 days post injury), when macrophages release cytokines and growth factors that recruit fibroblasts, keratinocytes and endothelial cells to repair the damaged tissues. The fibroblasts then begin to replace the provisional fibrin/fibronectin matrix with the new ECM. The ECM is largely constructed during the proliferative phase (˜day 3 to ˜2 weeks post injury) by the fibroblasts, which are cells that synthesize fibronectin and collagen. As granulation continues, other cell types, such as epithelial cells, mast cells, endothelial cells (involved in capillaries) migrate into the ECM as part of the healing process.

Stage 4 pressure ulcers can form in 8 hours or less, but take months or years to heal. Pressure ulcers are complicated wounds, which can include infection, exudates (watery mix of wound residue), slough (dead loose yellow tissue), black eschar (dead blackened tissue with a hard crust), hyperkeratosis (a region of hard grayish tissue surrounding the wound), and undermining or tunneling (an area of tissue destruction extending under intact skin). The general concept of undermining is illustrated in FIG. 5b, where there is a lateral extension of wound 205 under the surface of the intact skin. Although the illustration shows this undermining 207 being confined within the dermis, it typically includes loss of the deeper subcutaneous tissues (fat, muscles, etc.) as well.

The use of bandages and dressings in wound care very much depends upon the circumstances. In the case of a shallow wound (as in FIG. 5a), a single dressing may be placed over the wound. Whereas, in the case of a deep cavity wound, either acute or chronic, a primary dressing 250 may be inserted or packed into the wound, while a secondary dressing may be applied at the skin surface. Modern wound dressings are designed with a recognition that optimal wound healing requires an ideal environment, including adequate exudate absorption, moisture vapor control, prevention of secondary infection, protection from external forces, thermal insulation for tissue temperature control, and easy use without harming the wound or the surrounding tissues. In particular, it is now understood that optimal (quickest, with least scarring) wound healing requires a moist, but not wet, environment. Generally, there are different expectations for different types of dressings. For example, a deep tissue packing dressing, such as an alginate or a hydrofiber dressing are available as sheets or ropes, and are used to absorb exudates and fill dead spaces. Whereas a thin film dressing is placed over the wound at the skin surface, and is required to control the access of moisture and bacteria to the wound. A thin film dressing may also have an attached foam or alginate wafer to provide moderate absorption of exudates. More generally, the properties of a wound dressing are defined relative to the “occlusivity” of the dressing, relative to being generally impermeable to bacteria & water (keeping them from getting into the wound), but being either permeable or impermeable (basically semi-permeable) to water vapor, oxygen, and carbon dioxide.

While intact skin has a low moisture vapor transmission rate (MVTR) of 96-216 g/m² day, the MVTR of wounded skin is much higher, at 1920-2160 g/m² day. A moisture occlusive dressing (used for a dry wound) has a low MVTR (<300 g/m² day), a moisture retentive dressing has a mid-range MVTR (<840 g/m² day), and a permeable dressing (used for a wet wound) has a high MVTR (1600⁺ g/m² day). In many cases, a thin polymer film provides the barrier properties that determine the occlusivity, and thus control the interaction between the tissues and the outside environment. The MVTR of a film depends on the film thickness, the material properties, and the film manufacturing properties. The bacterial occlusivity of a film depends on the size of the pores (for example, <0.2 microns) and the thickness of the film. Larger pores (0.4-0.8 microns) will block bacteria depending on the organism and their number, the pore size, and the driving pressure. Thus, the film thickness must be co-optimized, as a thicker film will beneficially prevent bacterial penetration, but could then prevent sufficient moisture vapor transmission. Typical film dressings are thin elastic polyurethane sheets, which are transparent and semi-permeable to vapor, but have an outer surface that is water repellent. More generally, polyurethane is an exemplary moisture permeable film for a non-occlusive dressing is, and polyvinylidine chloride is an exemplary moisture impermeable film for an occlusive dressing. These continuous synthetic and non-toxic polymers films can be formed by casting, extrusion or other known film-making processes. The films thickness is less than 10 mils, usually of from 0.5 to 6 mils (10-150 microns). The film is continuous, that is, it has no perforations or pores that extend through the depth of the film. As a primary dressing, such film dressings are typically used for treating superficial wounds, including donor sites, blisters, or intravenous sites. For example, thin film dressings, such as Tegaderm from 3M, comprise a thin film with adhesive around the edge for attaching the dressing to the skin. A film layer can also be a component within a more complicated wound care dressing. For example, a foam dressing could combine an absorbent foam layer (to absorb exudates) with a thin film layer, to provide the needed occlusivity with the outside environment.

With the above understandings of wounds and wound care, it can now be appreciated that the light therapy bandage 300 of FIG. 7a can be equipped with a barrier layer or membrane 450, which can be a polyurethane thin film sheet which defines the occlusivity of bandage 300 relative to MVTR, bacterial access, and other properties. For example, film 450 could have a moderate MVTR appropriate for use with a moderately exuding wound. As such, it would allow a fair amount of moisture to evaporate out of the wound, in order to help optimize the wound moistness and healing. Barrier film 450 could either be permanent with bandage 300, or removable, and perhaps replaceable. However, it may not be sufficient to equip bandage 300 with barrier layer 450 attached to exit surface 490, as moisture could otherwise be trapped within the structure, as substrate 410 is likely too thick (1-3 mm) to allow effective moisture vapor transmission. The trapped moisture could condense within the bandage and then impair device function or become a breeding ground for bacteria. Thus, bandage 300 is provided with a multitude of vapor channels 460, nominally arranged between the flex circuitry 350, although they could pass through the flex as well, as long as they avoided the circuitry. Vapor channels 460 are nominally orthogonal to the large sheet-like surfaces of the bandage 300. However, vapor channels 460 could also run laterally with the bandage towards the edges of the bandage. The diameter and shape of the vapor channels should be such that the moisture vapor can exit relatively unencumbered through these channels, thus allowing the barrier properties to indeed be defined by layer 450.

Of course, as wound dressings are used in myriad ways and combinations, a circumstance may arise where light therapy bandage 300 is provided without a barrier layer 450, as that function is provided within another (primary) dressing. It should be understood that an absorbent layer, such as foam sponge or alginate pad could be attached to bandage 300, for example between the barrier layer 450 and the underlying tissue being treated. Of course, as bandage 300 is intended for use in light therapy, this absorbent layer should be nominally transparent as well. However, as some wound care dressings, such as those using alginates and hydrofibers, become transparent when wet, this is achievable. Additionally, and somewhat surprisingly, exudates, which principally comprise water, are generally transparent, or only moderately discolored. So, again, reasonable light transmission into the wound is possible.

For light therapy bandage 300 to be credible for wound care, it must be low profile, highly conformal, comfortable, and have a low cost manufacturability. Conformability is a particular concern, as the clinician may need to use the bandage 300 in a difficult location such as at the lower back/buttocks, or even within an undermined wound or body cavity. The use of surface mount light emitting diodes 372 mounted on flex circuitry 350 helps the design relative to cost, device profile, and conformability. Flex circuitry or ribbon cable is relatively thin (˜120 microns thick) and flexible. There are various types available, including polyamide and copper flex which can handle a “high” heat load, and polyester and aluminum which has a lower heat capacity, but does not require soldering and has a lower cost. As stated earlier, diodes 370 are nominally surface mount light emitting diodes (LEDs), which are compact (˜1 mm height) and which assembled onto the flex circuitry 350 with high-speed robotic equipment. It should be understood that diodes 370 could be other semiconductor optical devices, including laser diodes (such as VCSELs) and super luminescent diodes (SLDs). Diodes 370 can also use non-semiconductor light emitting technologies, such as organic LED technology.

As shown in FIGS. 6 and 7a, flex circuitry 350 is nominally assembled into light therapy bandage 300 as a series of adjacent strips or circuits. In particular, as shown in the top view of FIG. 7a, the multiple adjacent flex circuits 350 are nominally offset in the Y direction. As a result, the conformability of bandage 300 should be improved in the Y direction, as compared to using one wide sheet of flex circuitry. To further improve the conformability of the bandage 300, a serpentine flex circuitry 360 shown in FIG. 7b, with slits 362 or other features to reduce the rigidity, can be provided to further improve the flexibility of the bandage 300 in both the X and Y directions. Of course, the flex circuitry 350 could be spatially distributed in other ways within bandage 300, both regular and irregular, aside from the parallel arrangement of serpentine flex 360 shown in FIG. 7b, in order to enhance conformability.

FIG. 7c shows in cross section two potential constructions of the bandage 300 of the present invention. In the upper example, diodes 370 are assembled in a substrate 410, which includes material 470 and sheet material 420. Sheet material 420 can represent the flex circuitry, or the flex circuitry can be imbedded in sheet material 420. Support sheet material 420 can, for example, be a flexible solid polyurethane or silicone material. Material 420 can be either transparent or opaque, as long as it does not cover over the diodes 370 if opaque The top surface 485 of substrate material 420 may be provided with a top material 487, which could be reflective coating, such as an evaporated aluminum coating that would help keep stray light within the bandage 300 and tissue. Top material 487 could also be a thin, flexible mylar (polyester) sheet, with or without an outer evaporated reflective coating, which is laminated or otherwise fastened to sheet material 420. As mylar is a very tough material, an outer mylar layer would protect the bandage 300 from damage. However, as mylar has a very poor MVTR, the vapor channels 460 should penetrate through this material, to ensure moisture (and gases more generally) transmission.

The light-emitting portion of the LEDs is further encased or imbedded in a transparent material 470, through which the light 310 is transmitted towards the exit surface 490 and then a treatment area. Transparent material 470 could be fabricated (coated, molded, or cast) onto sheet material 420, which includes flex circuitry 350 and diodes 370. Alternately, substrate 410 could be fabricated by a process in which flex 350 is imbedded directly into material 470 without the use of a support sheet. Transparent material 470 can comprise a flexible transparent polyurethane, perhaps 0.5-1.0 mm thick. It is preferable, for robustness, cleanliness, and cost reasons that the exit surface of substrate 420 be continuous and smooth, without holes or perforations (aside from the vapor channels 460). Thus, light 310 is transmitted through the material 470, rather than having open-air channels through which light 310 travels to reach the exit surface 490.

It is noted the combined thickness of a top material 487, substrate 420 (with flex circuitry), and transparent material 470 could easily be 2-3 mm, which may impair conformability, even with the use of a serpentine flex and a pliable sheet materials. To further enhance conformability, transparent material 470 could be a polymer foam material, such as a solvent-coated polyurethane or a Dow Corning clear optical RTV. To minimize contamination issues, the foam cell size could be kept small (˜0.1 mm). Also, the foam could be fabricated or coated such that the exit surface 490 was generally continuous and smooth, with minimal open cells at the surface.

The lower illustration of FIG. 7c depicts an alternate cross-sectional construction of bandage 300, in which substrate 410 comprises an upper sheet material 420 (with the flex circuitry) and a transparent lower sheet material 420 having an exit surface 490, with transparent material 470 in-between. Transparent material 470 could again be an optically clear foam, but with lower sheet material 490 providing the continuity and smoothness. Alternately, transparent material 470 could be an optically transparent gel, which is encapsulated or sealed between the upper and lower sheet materials 420. Spacers 472 could be used to keep the overall thickness of the device 300 nominally constant, even if pressure is applied to the bandage 300. The upper and lower sheet materials could be welded towards the bandage edges and occasionally across the surface of the bandage, to seal the gel in and to keep the gel from collecting locally. The encapsulated transparent gel material could provide even greater conformability then does a bandage 300 with a foam core. In this instance, it is generally assumed that transparent gel material 470 must be kept out of the wounds, in order to not unintentionally alter the wound environment. As another alternative, bandage 300 could be provided with a transparent wound treatment gel, such as a hydrocolloid gel (Douderm from Convatec, for example) or an alginate wound gel, which is used to absorb or provide moisture to a wound, depending on the need. For example, a wound treatment gel could be provided with the design concept shown in the upper illustration of FIG. 7c, by applying the gel onto barrier membrane 450, between membrane 450 and the tissue (not shown). Such gels are not meant to be tacky, as wound dressings are designed to avoid adhesion with the wounded tissue, so as to avoid causing further damage. Of course, the encapsulated transparent gel material could likewise be a moisture absorbent gel, such as a hydrocolloid gel, so that some of the moisture vapor passing through barrier layer 450 is then trapped within the dressing 300.

Among the considerations in providing a light therapy device with on board light sources, such as multiple LEDs, is how to connect the diodes to a power source and how to control current and heat dissipation (thermal loading), while providing some measure of redundancy or robustness. If, for example, 100 LEDs are desired for a particular bandage size, they could all be connected in parallel. This arrangement would require the power source to provide a large current. For example, for 100 LEDs at 20 mA each, 2 amps of current would be needed. If one LED went open circuit, the extra 20 mA of current would be divided among the other 99 LEDs and would not generally be a problem. However, if one LED shorted internally, the current would be diverted to the short, and all of the LEDs would go dark. The bandage would no longer be useful.

Alternately, if the LEDs were connected in series, a large source voltage would be needed. For example, if the forward voltage drop for a near IR LED is 1.8 volts, 100 LEDs in series would require a 180V source—a possible high voltage hazard for the patient. If one LED shorted internally, the extra current would be shared among the remaining 99 LEDs. If one LED went open circuit, all the LEDs would go dark. Again, the bandage would be rendered useless.

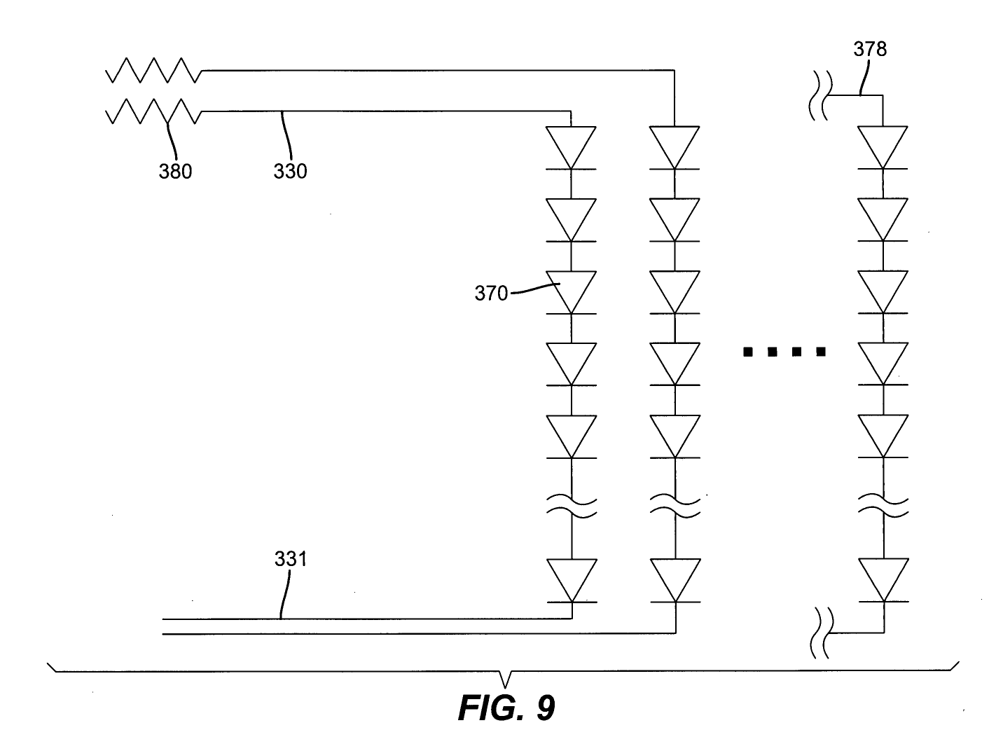

However, a series-parallel arrangement of LEDs would be a preferred embodiment, and is practical from a power supply, LED variation, and a robustness standpoint. The general concept is described with respect to FIG. 9. Again assume that 100 LEDs are required. One possible arrangement is to have 10 parallel strings of LEDs, with 10 LEDs in series in each string. Assume the nominal forward voltage drop is 1.8V. Ten series LEDs (diodes 370) would nominally require 18V, a voltage that doesn't represent a shock hazard. Then each series string or grouping 378 would nominally require 20 mA. A constant current source could be used, but often a voltage source is used for cost and complexity reasons. Using a supply voltage of 20V, approximately 18V is dropped across the LEDs and the remaining 2V can be dropped across a current limiting resistor (380) of 100 ohms to limit the current to 20 mA. This resistor 380 will dissipate 40 mW in heat, which may not be desirable in the bandage. However, the current limiting resistors 380 can be located remotely, for example with a power supply in controller 320 or in intermediate 325, such that the heat can easily be handled. Connective circuitry 330 would then supply power to the diodes 370 in a series string or grouping 378. Each of the 10 parallel strings would be handled similarly, each with a current limiting resistor 380. The total power dissipation from the resistors would be ten times 40 mW or 400 mW. Ten parallel strings, each requiring 20 mA, requires the power source to supply 200 mA. The return current paths 331 are shown as separate for each string in FIG. 9, but could be combined as a single path or ground plane.

Now, if a single diode 370 happens to short internally, the voltage across that string drops from 18V to 16.2V. The remaining 1.8V will be dropped across the 100 ohm limiting resistor, and the current will now be 38 mA in that string. If the LEDs have good heatsinking capability, they can easily stand this increased current, and the total light out of the bandage will increase on the order of 8%. If one of the LEDs becomes open circuited, the entire string of 10 LEDs goes dark, but the rest of the strings stay lit and the light output from the bandage will drop on the order of 10%.

Note that not all commercially available LEDs have the same forward voltage drop or the same voltage vs. current (VI) characteristics. For example, some may have a drop of 1.75V and others of 1.85V. Thus a series string of N randomly selected diodes will tend to average out the variations, thus precluding a selection process. In addition, it may be advantageous to make one LED diode 370 in each string a red LED. Red light has also been shown to be beneficial in healing and it can be an indicator that the bandage is on and functioning normally. Red LEDs typically have a lower forward voltage than near IR LEDs, on the order of 1.5V. However, this difference would tend to get washed out by the rest of the LEDs. In this case, the forward voltage drop for a string of 9 IR LEDs (1.8V each) and one red LED (1.5V) would be 17.7V. The LEDs could tolerate the small current increase this would cause, or the resistance of the current limiting resistor could be raised slightly. Therefore, a red LED could easily be substituted in each string, if desired, without requiring a design change.

Each IR LED is assumed to have a forward voltage drop of 1.8V and a current of 20 mA. The power dissipation is the product of voltage and current, or about 36 mW per LED. For 100 LEDs, it would be 3.6 W. Assuming about 25% conversion efficiency to light, about 2.7 W will be dissipated as heat. The bandage could become warm to the touch but not so hot that you could not keep your hand in contact with it. For comparison, a small tungsten bulb typically used in Christmas candles and other decorations is 7.5 W. It may well be advantageous to keep the wound area warm, but not hot. However, the light efficiency of the LEDs drops rapidly as they get hot and from an LED efficiency and optical power standpoint, the cooler the better. LEDs are typically rated for at least 50° C. Room temperature is 23° C., skin temperature is about 30° C. and internal body temperature is 37° C. The maximum temperature recommended for a hot tub (total body immersion) is around 42° C. As long as the bandage stays below 42° C., it should not be harmful. The body itself can provide substantial heat sinking properties for the bandage, especially if it is running at about 35° C. Using this series-parallel approach to drive the diodes 370, a portion (˜20% or more, depending on the number of LEDs) of the heat should be generated and dissipated in the remote current limiting resistors 380 rather than originating at the diodes 370 in bandage 300. However, additional heat sinking properties can be provided in the bandage itself to ensure maximum light output from the bandage for optimal healing conditions. Alternately, a quantity of current limiting resistors could be provided in bandage 300, if additional heat was wanted.

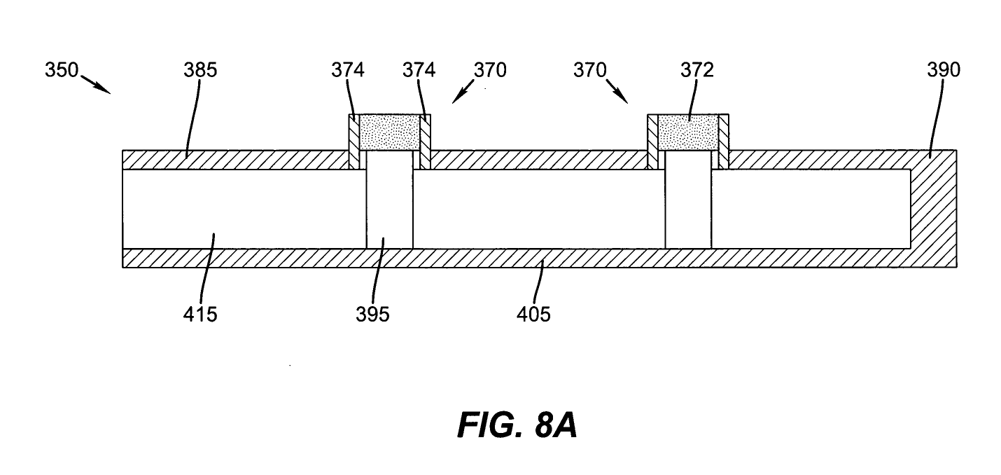

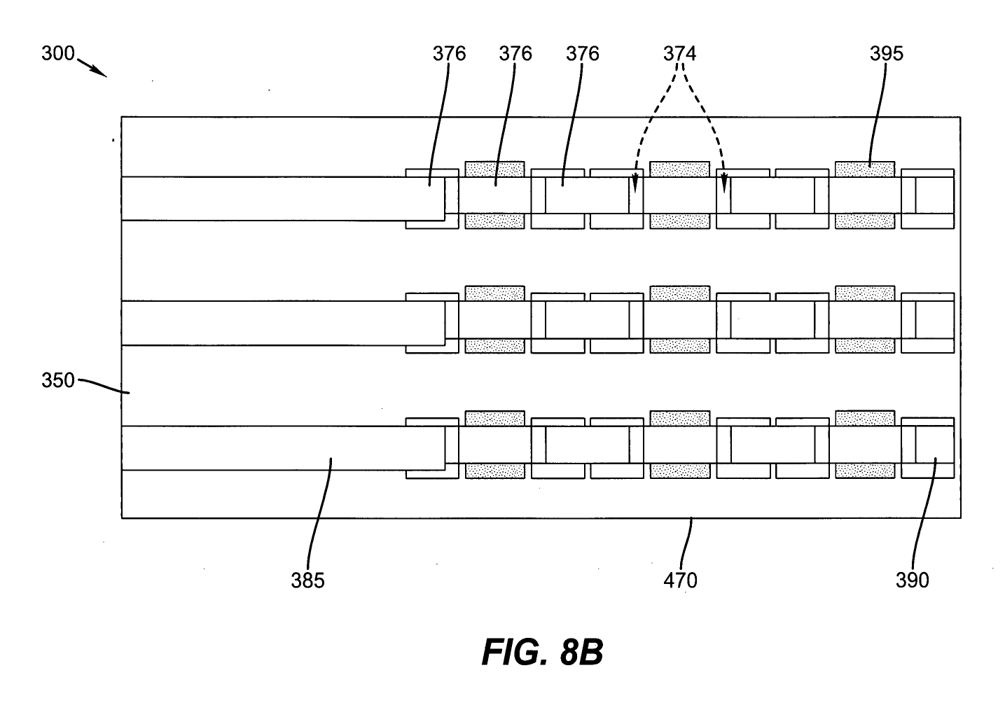

FIG. 8a is a side view of the bandage 300, showing two LEDs 350 in series to illustrate how the flex circuit 350 might be constructed. The flex circuit 350 is preferably constructed using a flexible internal insulating material with metal conductors (385 and 405) on each side, although the conductors may be confined to one side. The insulating material 415 could be a polyamide such as Kapton, while the conductors could be made out of copper. This construction would allow soldering of the LEDs to the flex circuit for maximum electrical and thermal conductivity. Alternately, the flexible material could be polyester and the xsconductors made out of aluminum. This choice would typically be half the cost of the first method but may not have quite as good a performance, especially from a thermal standpoint, as soldering can not be used. However, thermally conductive adhesives are available, as well as good electrical and thermally conducting adhesives, such as silver epoxy. The choice may be driven by the size of the bandage and the number of LEDs required. Large areas require more LEDs to provide a given irradiance in mW/cm² of light, and large numbers of LEDs will generate heat, which must be dealt with efficiently.

As shown in FIG. 8a, the address trace 385 is on the top surface and will face the wound (not shown). LEDs 370, which are preferably surface mount diodes 372, are soldered or bonded at electrodes 374 to pads 376 connecting to these address traces. Typical LED chips are soldered or adhesively attached to a conductor on a substrate, and a wire bond is made to the electrode on the top surface. The wire is very thin and can be a source of failure if subjected to flexing and stretching during use. A surface mount LED is much more rugged because everything is encapsulated. Furthermore, surface mount devices are easily utilized in high-speed assembly processes. The underside (opposite to the light emission) of each surface mount LED is typically a ceramic, such as alumina (aluminum-oxide) or beryllia, with a high thermal conductivity, but low electrical conductivity. Accordingly, a specialized flex 350 would provide a hole (thermal via 395) under each LED, which passes through the flexible insulator 415 to the metal ground plane 405 on the opposite side. The thermal via 395 is plated or filled such that a thermal path is provided to the ground plane. The LED is soldered or bonded to the thermal via 395, and then heat generated by the LED can be quickly conducted away from the LED and spread out into the ground plane 405 that is away from the patient. Ground plane 405 could be exposed to air for conductive and convective cooling. Because the ground plane is thin, typically a few thousandths of an inch, it remains flexible. The address trace 385 continues along the top surface from LED to LED connecting each in series. At the end of the LED string or grouping, an additional via (or group of vias for redundancy) can provide electrical connection to the ground plane for return of current to the power supply. These electrical return vias 390 themselves can be filled, providing additional heat and electrical capacity. Metallic silver could be used to plate the conductors and fill the vias to provide electrical and thermal conductivity. These vias would have a mirror-like reflective surface to reflect the light toward the skin of the patient and prevent light from leaking out the back of the bandage. Alternately, silver epoxy or thermal epoxy could be used. Silver or thermal epoxy underneath the LEDs 372 could be used to affix the LED to the flex and would naturally fill the thermal vias 395. The address traces 385 and the ground plane 395 can be made serpentine, spiral, or finger-like to improve flexibility and conformability.

The flex circuit of FIG. 8a can be encapsulated in a polymer material, such as a polyester or polyamide (not shown), so that it can be handled separately, while protecting the circuit elements. The flex circuit can then be imbedded in the transparent material 470 (such as the exemplary clear polyurethane or silicone rubber) with the total thickness being 2 mm or less.

FIG. 8b is a top view of a portion of bandage 300 showing three parallel strings of surface mount LEDs 372, each with three LEDs in series. The current flows along the address trace 385 through the first LED 372, and continues along the trace through each successive LED 372. At the end of the series string of LEDs, an electrically conductive return via 390 is provided to establish an electrical connection to the ground plane and a return current path. The conductor plane on the backside of the flex 350 can perform multiple roles: current conductor, EMI shield, flexible heat sink, and mirror. Together with the address line, the ground plane forms a microstrip transmission line, allowing transmittance of high frequency signals while minimizing radiated electromagnetic energy that could interfere with nearby medical equipment. If necessary, the vapor channels could be routed to help carry heat away from the ground plane.

Each LED shown in FIG. 8b is a surface mount device 372 made for high volume pick-and-place machines, and has electrode connections 374 at either end. These electrodes 374 are placed on pads 376 connected to the address traces 385 and the LEDs 372 are soldered or bonded to the pad to make electrical contact. In addition, as previously described, each LED is attached to a via pad directly beneath it which provides thermal contact with ground plane on the opposite side of the flexible insulator from the LED. The thermal via 395 is a plated through hole in the insulator which can filled with solder or electrically or thermally conductive adhesive.

The diode groupings 378 can be distributed within bandage 300 in a multitude of ways. Parallel groups or strings can be routed in a spatially parallel fashion, so that an area of tissue tends to receive light from multiple groups, thus enhancing redundancy. Alternately, the groupings 378 can be spatially patterned, as suggested in FIG. 7a, so that controlling which groups are on or off can provide spatial addressing to the tissue. In this way, the spatial addressing suggested by FIGS. 4a-4c could be realized. Other parameters, such as frequency or intensity could then be controlled in a spatially variant manner. As one explicit example of this, FIG. 7e depicts a design for bandage 300 wherein the flex circuitry is routed concentrically within the device. By having a set of concentric flex 365, bandage 300 can provide spatial addressing without requiring passive or active matrix addressing, as is used in imaging devices. The concentric flex 365 can, on a local scale, have a serpentine pattern, much as shown in FIG. 7b.

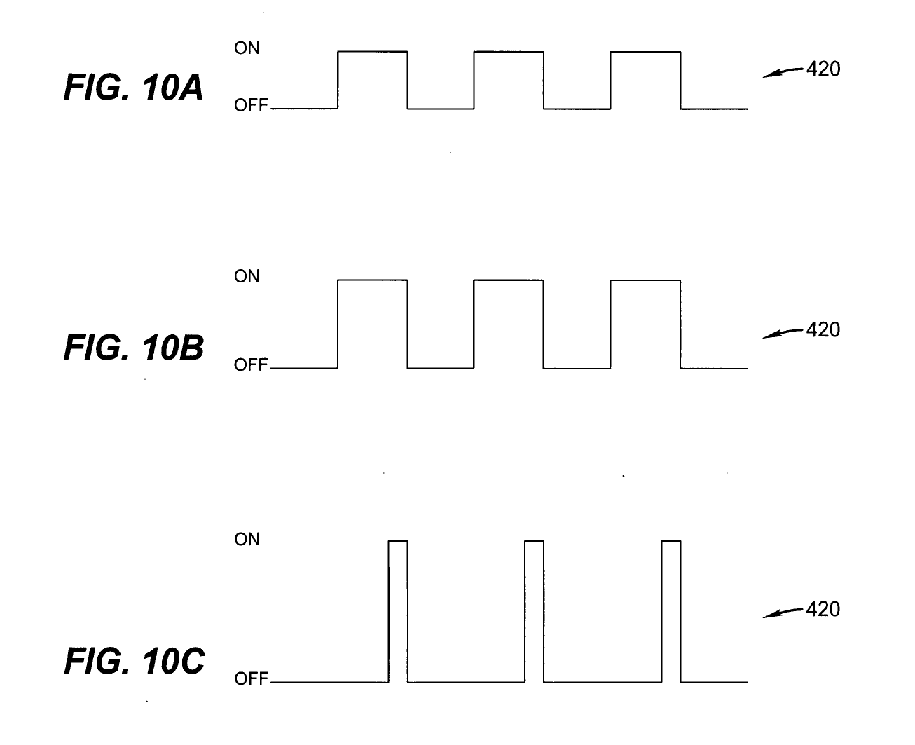

It may be desirable or even necessary to increase the peak optical power of the LEDs in the bandage or alternatively, to reduce the average power dissipated as heat. Pulsing the LEDs, rather than running them continuously can also enhance heat capacity and control in the bandage 300. FIG. 10a shows an LED current waveform 400 with a 50% duty cycle squarewave. The horizontal axis represents time and the vertical axis can represent either instantaneous current or light output. The current or light is switched on and off. Since electrical power is being delivered only half the time to the LED, the heat dissipation is cut in half. This method reduces heat load to the bandage, while allowing the LEDs to operate at a lower temperature and at a greater optical (quantum) efficiency. For example, a bandage running with continuous current might be running at a 10 mw/cm² output, where a bandage with the same current at a 50% duty cycle, might have a peak pulse power of 10 to 12 mW/cm² and an average power of 5 to 6 mW/cm². In this case, as exposure is equal to intensity times time, to achieve the same exposure of the wound, the exposure time would have to be approximately doubled.

However, it may be that the would healing efficacy has a light power threshold and that as long as the peak pulse power is above that threshold, improved healing will occur. FIG. 10b shows another 50% duty cycle waveform where the peak current or light is approximately twice the previous waveform. In this case the average light power and heat dissipation would be the same as the current level of waveform (a) but with constant current. The per second exposure would also be the same as a constant current at half the level. An approach such as this would allow double the peak light power while maintaining the same average light power and heat dissipation as a continuous DC current at half the peak level of (b). It is noted that LEDs are capable of being run at 10 times the rated DC current, at a small duty cycle, often 10% or less. FIG. 10c shows a waveform with high peak pulse power and low duty cycle. Running an LED at 10 times the rated DC current does not necessarily give 10 times the peak light out. The results vary widely by LED material and manufacturer. Peak light output typically runs between 3 times and 10 times the continuous light output, depending on the LED type. Moreover, a combination of low duty cycle and a high peak power can actually deliver more light output than the same diode run CW at the average current for the same time frame. Thus, varying the duty cycle and peak current amplitude allows tradeoffs to be made in peak optical power and/or average heat dissipation, with particular benefits for duty cycles of <50% (more time off than on). These tradeoff choices may vary depending on the size of the area to be irradiated and the number of LEDs needed.

In the field of light therapy, there is significant uncertainty as to whether light therapy is best applied with continuous or pulsed light, or even a combination thereof. Different operating conditions (CW or pulsed) are attributed to various medical conditions, depending on wavelength, intensity, and patient responsiveness, by different researchers. Suggested operational frequencies vary from CW (continuous wave) to a few Hz to 8 kHz, but with stated or implied 50% duty cycles. Bandage 300 can be operated, relative to discussion related to FIGS. 10a-10c, such that the operational frequency matches a recommended treatment rate, but with a duty cycle that enhances heat dissipation. Bandage 300 can also be operated at a frequency so fast that the tissue responds to the light as if it were CW, while the diodes 370 experience the previously described low duty cycle pulsed operation and reduced thermal loading.

Taken together, the various approaches towards the electrical design, including the use of a combination series-parallel circuitry with remote current limiting resistors, flex circuitry design with thermal vias and a common ground plane, and pulsed current control, can provide useful approaches for thermal management for the light therapy bandage 300. These approaches can be used individually, or in combination, to minimize and control the thermal loading within the device. These thermal management and control means can also include one or more thermal sensors (such as a thermistors) located in the bandage 300 or in the controller 320 to detect thermal loading, overloading, or failure, and a shut down mechanism to deactivate the bandage. By comparison, the prior art devices allow significant heat to originate in the light therapy dressings, and then require cumbersome heat sinks, heat dissipating layers, or cooling channels to help dissipate the heat.

While the bandage 300 has been principally described with flex circuitry (350 or 360) and surface mount LEDs 372, this is not a requirement. For example, organic LEDs, polymer LEDs, thin film electroluminescent (TFEL) emitters, and other patternable light source technologies could be used instead. Admittedly, these technologies have issues relative to efficiency and intensity, wavelength, moisture shortened lifetimes, and toxicity to overcome. However, assuming these issues are resolved, a light therapy device 300 with patterned emitters that is overlaid with a flexible transparent material (such as a polymer sheet or foam), provided with a barrier membrane and vapor channels, and electrically designed and driven to minimize thermal loading, could be useful as well.

As previously described, the flex circuitry is to be fastened or imbedded into a substrate 410, which includes transparent material 470. A protrusion of the diodes 370 into these materials will be provide significant frictional resistance for the flex circuitry, relative to it being pulled out of the end of bandage 300. However, outer protective layers of flex circuitry (350 or 360), whether of polyamide or polyester, tend to be smooth, which could limit the strength of the chemical and mechanical bonding of the flex and the adjacent materials (420 and 470). To enhance the mechanical integrity of device 300, the outer surfaces of the flex circuitry could be mechanically or chemically scuffed or roughened to provide shallow abrasions or the equivalent, to enhance the subsequent bonding strength and spatial consistency. Likewise, if the bandage 300 was torqued or twisted, the flex circuitry could twist within the bandage and potentially degrade its operation or mechanical integrity. Again roughening the outer surfaces of the flex would be a preventive measure. There are other design approaches as well, such as imbedding reinforcement threads in substrate 410 (per FIG. 7a, in the Y direction, or diagonally across the bandage), much as is done with fiberglass-reinforced tape. The mechanical integrity of bandage 300 could then be significantly enhanced, with minimal impact on the conformability.

It was previously mentioned that wounds could be complex and require complex approaches to treatment. For example, FIG. 5b depicts a wound with full thickness skin loss, with the wound 205 penetrating the epidermis 220 and the dermis 230, as well as a portion of the subcutaneous tissue 240. As shown in FIG. 5b, light therapy device 300 is provided as a secondary dressing, with a primary wound dressing 250 packed into the wound 205. In this illustration, therapeutic light (λ) is shown propagating through the primary dressing to be incident on the deeper tissues. As some primary wound dressings used for wound packing, such as hydrofiber (Convatec Aquacel) and alginate dressings, can become reasonably transparent when wet, this is plausible. However, some packing dressings, such as the KCI wound care vacuum sponge, are not presently optically transparent. In such cases, it may be desirable to route the therapeutic light into the wound. As shown in FIG. 5c, light therapy device 300 could have bandage extensions 340 that could be inserted into the wound 205. Correspondingly, FIG. 7d depicts a device 300 with flex circuitry bearing diodes 370 routed into bandage extensions 340. These bandage extensions could be constructed with diodes 370 facing both ways (towards the “top” and bottom”) to assist multi-directional light therapy.

It should be understood that the light therapy device of the present invention has been described in a general way, and that various modifications and additions are anticipated that could be made. For example, device 300 could include an internal light diffusion layer 480, as generally shown in FIG. 7a. As another example, ongoing research into light therapy has suggested that it can be advantageous to illuminate the tissue being treated with polarized light, as compared to non-polarized light. Therefore it may be beneficial to equip the light therapy device 300 with a polarizing film Within the substrate structure 410, if the diodes 370 do not emit polarized light.

Additionally device 300 could have antibiotic properties, including the possible use of a transparent anti-biotic silver, as is described in copending, commonly-assigned, French Patent Application 0508508, filed Aug. 11, 2005 by Y. Lerat et al. Device 300 could also have added bio-sensing capabilities or topical agents that encourage epithelialization or other tissue healing activities, to possibly amplify the effects of light therapy. In the case of bio-sensing, the bio-sensor features might detect a bio-physical or bio-chemical condition of the treatment area, which can then be used as input to guide further treatments. For example, the biosensors might detect the presence or absence of certain pathogens or enzymes associated with infections, or other enzymes and proteins associated with healing. Light guide device 300 could also be equipped with a sensing means that changes color relative to time to indicate the time (or amount of exposure) and thereby indicates an end to a given therapy session. For example, biosensors could be used to look for bio-chemical indications of the effective dosage applied. Alternately, optical sensors could detect the backscattered light as measure of the optical dosage delivered. The end of session control could then be manual or automatic.

Light therapy device 300 may also have adhesive layers on an inner surface that might help to attach the device directly onto the tissue (outside the wound), or to other bandage elements. Alternately, adhesive layers could represent other types of attachment means, such as Velcro, which could be used to fasten the light therapy device 300 to other bandage elements. Device 300 has been generally described as incorporating a barrier membrane 450 to control bacterial transfer. As noted, this barrier could potentially be replaceable. Indeed, it could be provided as a hygienic sleeve instead, which would slide over a significant portion of the device.

The light therapy device 300 of the present invention has been principally considered with respect to the anticipated use in treating human patients for light therapy and PDT. Certainly, the device 300 could be used for other purposes, of which veterinary care is the most obvious. A potential use for industrial or agricultural purposes is unclear, and yet the device 300 could be used to deliver light to an irregular area in which there is relevant concern for moisture in the area, and/or thermal loading in the area of application or the device itself.

The invention has been described in detail with particular reference to a presently preferred embodiment, but it will be understood that variations and modifications can be effected within the scope of the invention. The presently disclosed embodiments are therefore considered in all respects to be illustrative and not restrictive. The scope of the invention is indicated by the appended claims, and all changes that come within the meaning and range of equivalents thereof are intended to be embraced therein.

### PARTS LIST

10 fiber-optic pad
12 fiber-optic cable
14 enclosure
16 connector
20 controller
50 light therapy device
55 light therapy bandage
60 straps
65 housing
75 light emitters (LEDs or laser diodes)
80 clear windows
85 flexible drive circuitry
90 battery
95 heat sinks
100 heat dissipating layer
105 shell
110 liner
115 diffuser
120 exposed tacky surface
130 substrate
135 reflector
140 bubbles
145 front cover
147 back cover
155 cooling channel
157 secondary cooling channel
200 tissue
205 wound
207 tunneling or undermining
210 skin surface
220 epidermis
225 stratum corneum
230 dermis
240 subcutaneous tissue
250 primary wound dressing
300 light therapy bandage (or dressing)
305 light therapy areas
310 light
320 controller
325 intermediate
330 connective circuitry
331 return current paths
340 bandage extensions
350 flex circuitry
360 serpentine flex
362 slits
365 concentric flex
370 diodes
372 surface mount LED
374 electrodes
376 solder/adhesive pads
378 diode groupings
380 resistors
385 address trace
390 electrical return via
395 thermal via
400 current or light intensity waveforms
405 ground plane
410 substrate
415 insulating material
420 sheet material
450 barrier membrane
460 vapor channels
470 transparent material
472 spacers
480 diffuser
485 top surface
487 top material
490 exit surface

1. A light therapy bandage for delivering light energy to treat medical conditions in tissues comprising:
a plurality of flexible sheet circuitry, each of which is fabricated with a serpentine pattern and each of which is provided with one or more surface mounted light emitting devices that emit said light energy, wherein said flexible sheet circuitry is assembled into a substrate;
a flexible transparent material included within said substrate, which is applied such that said surface mounted light emitting devices are imbedded in said flexible transparent material;
a semi-permeable transparent membrane attached to said flexible transparent material, which controls the flow of moisture and moisture vapor to and from said tissues;
a plurality of vapor channels which extend from said semi-permeable transparent membrane and through said substrate; and
wherein said light energy passes through said substrate and said semi-permeable membrane to be incident to said tissues, and wherein said moisture vapor passes through said semi-permeable membrane and said vapor channels and into the surrounding environment.

2. A light therapy bandage as in claim 1 wherein said semi-permeable transparent membrane is a polyurethane based thin film.

3. A light therapy bandage as in claim 1 wherein said semi-permeable transparent membrane is removable.

4. A light therapy bandage as in claim 1 wherein said semi-permeable membrane minimizes the passage of bacteria and controls the rate of moisture vapor transmission.

5. A light therapy bandage as in claim 1 wherein said vapor channels are nominally orthogonal to a plane nominally common with said flexible sheet circuitry.

6. A light therapy bandage as in claim 1 wherein said surface mounted light emitting devices emit red light, infrared light from the spectral range of 700-1300 nm, or some combination thereof.

7. A light therapy bandage as in claim 1 wherein said surface mounted light emitting devices are LEDs, laser diodes, SLDs, or other compact light emitting devices, or combinations thereof.

8. A light therapy bandage as in claim 1 wherein said flexible sheet circuitry is fabricated with an encapsulating polymer material, such as a polyamide, and wherein said flexible sheet circuitry has outer surfaces which are roughened by appropriate means, such as mechanical abrasion or chemical etching.

9. A light therapy bandage as in claim 1 wherein an outer surface of said substrate, which is oriented closest to said surrounding environment, has a layer of polyester (mylar) film applied to it.

10. A light therapy bandage as in claim 1 wherein said substrate further comprises an arrangement of reinforcement threads to improve the mechanical integrity of said light therapy bandage.

11. A light therapy bandage as in claim 1 wherein said flexible transparent material comprises a solid sheet like polymer material, such as a polyurethane.

12. A light therapy bandage as in claim i wherein said flexible transparent material comprises either a foam or a gel.

13. A light therapy bandage as in claim 12 wherein a surface of said foam in proximity to said semi-permeable transparent membrane is processed to be nominally smooth and continuous.

14. A light therapy bandage as in claim 12 wherein a thin polymer sheet is applied to between said gel and said semi-permeable membrane, to seal said gel within said light therapy bandage.

15. A light therapy bandage as in claim 12 wherein said gel is a water absorbing gel, such as a hydrocolloid gel.

16. A light therapy bandage as in claim 1 wherein an optical diffuser, or a volume with optical diffusing properties, is provided within said substrate, between said surface mount light emitting diodes and said semi-permeable transparent membrane.

17. A light therapy bandage as in claim 1 which further comprises a thermal control means for said light therapy bandage, comprising, either individually or in combination, the use of remote current limiting resistors, thermal vias within said flex circuitry for extracting heat from said surface mounted light emitting devices, and low duty cycle operation of said surface mounted light emitting devices.

18. A light therapy bandage as in claim 1 wherein an intermediate bandage portion is attached to said substrate as an interface between said substrate and a controller.

19. A light therapy bandage as in claim 1 wherein operation of said bandage in provided by a controller.

20. A light therapy bandage as in claim 1 wherein said surface mount light emitting diodes are connected by said flex circuitry to facilitate localized spatial pattern control of said light energy application.

21. A light therapy bandage as in claim 1 wherein an intermediate bandage portion is attached to said substrate as an interface between said substrate and a controller.

22. A light therapy bandage as in claim 1 wherein said light therapy bandage is used as a primary dressing or bandage for treatment of said medical condition.

23. A light therapy bandage as in claim 1 wherein said light therapy bandage is a secondary dressing or bandage, which is used in conjunction with a primary dressing or bandage for treatment of said medical condition.

24. A light therapy bandage for delivering light energy to treat medical conditions in tissues comprising:
a plurality of flexible sheet circuitry, each of which is provided with one or more surface mounted light emitting devices that emit said light energy, wherein said flexible sheet circuitry is assembled into a substrate;
a flexible transparent material included within said substrate, which is applied such that said surface mounted light emitting devices are imbedded in said flexible transparent material;
a semi-permeable transparent membrane attached to said flexible transparent material, which controls the flow of moisture and moisture vapor to and from said tissues;
a plurality of vapor channels which extend from said semi-permeable transparent membrane and through said substrate;
wherein said light energy passes through said substrate and said semi-permeable membrane to be incident to said tissues, and wherein said moisture vapor passes through said semi-permeable membrane and said vapor channels and into the surrounding environment; and
wherein said flexible transparent material comprises an optically clear foam or gel.

25. A light therapy bandage as in claim 24 wherein said flexible sheet circuitry is fabricated with a serpentine pattern.

26. A light therapy bandage as in claim 24 which further comprises a thermal control means for said light therapy bandage, comprising, either individually or in combination, the use of remote current limiting resistors, thermal vias within said flex circuitry for extracting heat from said surface mounted light emitting devices, and low duty cycle operation of said surface mounted light emitting devices.

27. A light therapy bandage as in claim 24 wherein said gel is a water absorbing gel, such as a hydrocolloid gel.

28. A light therapy bandage as in claim 24 wherein said semi-permeable transparent membrane is a polyurethane based thin film.

29. A light therapy bandage as in claim 24 wherein said semi-permeable membrane minimizes the passage of bacteria and controls the rate of moisture vapor transmission.

30. A light therapy bandage for delivering light energy to treat medical conditions in tissues comprising:
a plurality of flexible sheet circuitry, each of which is provided with one or more surface mounted light emitting devices that emit said light energy, wherein said flexible sheet circuitry is assembled into a substrate;
a flexible transparent material included within said substrate, which is applied such that said surface mounted light emitting devices are imbedded in said flexible transparent material;
a semi-permeable transparent membrane attached to said flexible transparent material, which controls a flow of moisture and moisture vapor to and from said tissues;
a plurality of vapor channels which extend from said semi-permeable transparent membrane and through said substrate;
a thermal control means for said light therapy bandage, comprising, either individually or in combination, remote current limiting resistors, thermal vias within flex circuitry for extracting heat from said surface mounted light emitting devices, and low duty cycle operation of said surface mounted light emitting devices; and
wherein said light energy passes through said substrate and said semi-permeable membrane incident to said tissues, and wherein said moisture vapor passes through said semi-permeable membrane and said vapor channels and into a surrounding environment.

31. A light therapy bandage as in claim 30 wherein said flexible sheet circuitry is fabricated with a serpentine pattern.

32. A light therapy bandage as in claim 30 wherein said semi-permeable transparent membrane is a polyurethane based thin film.

33. A light therapy bandage as in claim 30 wherein said semi-permeable membrane minimizes passage of bacteria and controls a rate of moisture vapor transmission.

34. A light therapy bandage as in claim 30 wherein said flexible transparent material comprises a solid sheet like polymer material, such as a polyurethane.

35. A light therapy bandage as in claim 30 wherein said flexible transparent material comprises either a foam or a gel.

36. A light therapy bandage for delivering light energy to treat medical conditions in tissues comprising:
a plurality of flexible circuits, each of which comprises one or more surface mounted light emitting devices that emit said light energy, wherein said flexible circuits are assembled into a substrate;
a flexible transparent material included within said substrate, wherein said surface mounted light emitting devices are imbedded in said flexible transparent material;
a thermal control means for said light therapy bandage, comprising, either individually or in combination, remote current limiting resistors, thermal vias within said flexible circuits for extracting heat from said surface mounted light emitting devices, and low duty cycle operation of said surface mounted light emitting devices; and
wherein said light energy passes through said substrate incident to said tissues.

37. A light therapy bandage as in claim 36 wherein said flexible circuits are fabricated with a serpentine pattern.

38. A light therapy bandage as in claim 36 wherein said flexible transparent material comprises a solid sheet like polymer material.

39. A light therapy bandage as in claim 36 wherein said flexible transparent material comprises either a foam or a gel.

40. A light therapy bandage for delivering light energy to treat medical conditions in tissues comprising:
a plurality of flexible sheet circuitry, each of which is provided with one or more surface mounted light emitting devices that emit said light energy, wherein said flexible sheet circuitry is assembled into a substrate;
a flexible transparent material included within said substrate, wherein that said surface mounted light emitting devices are imbedded in said flexible transparent material;
a semi-permeable transparent membrane attached to said flexible transparent material, which controls a flow of moisture and moisture vapor to and from said tissues;
a plurality of vapor channels which extend from said semi-permeable transparent membrane and through said substrate;
a thermal control means for said light therapy bandage to minimize a thermal load originating within said light therapy bandage; and
wherein said light energy passes through said substrate and said semi-permeable membrane incident to said tissues, and wherein said moisture vapor passes through said semi-permeable membrane and said vapor channels.

41. A light therapy bandage as in claim 40 wherein said thermal control means comprises, either individually or in combination, remote current limiting resistors, thermal vias within said flex circuitry for extracting heat from said surface mounted light emitting devices, or low duty cycle operation of said surface mounted light emitting devices

42. A light therapy bandage for delivering light energy to treat medical conditions in tissues comprising:
a plurality of light emitting devices, which are interconnected by drive circuitry, wherein said light emitting devices and said drive circuitry are assembled into a substrate;
a flexible transparent material included within said substrate, wherein that said light emitting devices are imbedded in said flexible transparent material;
semi-permeable transparent membrane attached to said flexible transparent material, which controls a flow of moisture and moisture vapor to and from said tissues;
a plurality of vapor channels which extend from said semi-permeable transparent membrane and through said substrate; and
wherein said light energy passes through said substrate and said semi-permeable membrane incident to said tissues, and wherein said moisture vapor passes through said semi-permeable membrane and said vapor channels.

43. A light therapy device for delivering light energy to treat medical conditions in tissues comprising:
a substrate comprising a plurality of light emitting devices, imbedded in a flexible transparent material;
a semi-permeable transparent membrane attached to said flexible transparent material, which controls a flow of moisture and moisture vapor to and from said tissues;
a plurality of vapor channels which extend from said semi-permeable transparent membrane and through said substrate;
a controller which controls operation of said light therapy device;
a thermal control means for said light therapy device to minimize the thermal load originating within said light therapy bandage; and
wherein said light energy passes through said substrate and said semi-permeable membrane incident to said tissues, and wherein said moisture vapor passes through said semi-permeable membrane and said vapor channels and into the surrounding environment.

44. A light therapy device as in claim 43 wherein said thermal control means comprises, either individually or in combination, remote current limiting resistors, thermal vias within said flex circuitry for extracting heat from said surface mounted light emitting devices, and low duty cycle operation of said surface mounted light emitting devices

45. A light therapy device as in claim 44 wherein at least a portion of said current limiting resistors are located in said controller.
